(19) United States
(12) Patent Application Publication
(10) Pub. No.: US 2022/0079744 A1
(43) Pub. Date: Mar. 17, 2022

(54) SCLERAL FIXATION DEVICE FOR INTRAOCULAR LENS SUPPORT AND SURGICAL METHODS FOR THE INSERTION OF THE DEVICE

(71) Applicant: Jose Antonio Arrieta, Buenos Aires (AR)

(72) Inventor: Jose Antonio Arrieta, Buenos Aires (AR)

(21) Appl. No.: 17/474,104

(22) Filed: Sep. 14, 2021

(30) Foreign Application Priority Data
Sep. 14, 2020 (AR) 20200102552

(51) Int. Cl. A61F 2/16; A61F 2/14

(52) U.S. Cl. CPC A61F 2/16; A61F 2002/1683; A61F 2/15

(57) ABSTRACT

A scleral fixation device for intraocular lens support (Arrieta's Holder) including: a receptacle and a plurality of Arrieta's Supports from the receptacle to the sclera, and surgical methods of inserting the device.

### FIELD OF THE INVENTION

The present invention refers to a scleral fixation device for intraocular lens support (hereinafter it will be called Arrieta's Holder) with scleral support to treat the consequences of complicated cataract surgery, ocular trauma or a weakness of the capsular support, in which case they are local anatomical alterations or secondary to systemic diseases and their placement method.

### DESCRIPTION OF PRIOR ART

### Introduction

Cataract surgery is one of the most commonly performed surgical procedures in the world. Ideally, the cataract is removed and an intraocular lens (IOL) is implanted into the capsular bag in a single process. However, the absence of adequate capsular support requires alternative approaches, including implantation of anterior chamber IOL lenses with either scleral or iris fixation.

Anterior chamber intraocular lenses (IOLs) provide good results, but complications have been reported, such as cystoid macular edema, glaucoma, uveitis, peripheral anterior synechiae, pupillary ovalization, iris atrophy, hyphema, irreversible loss of endothelial cells, and corneal decompensation. Therefore, in recent years techniques have been developed that aim to reduce the incidence of complications, thus arising the fixation of intraocular lenses to the iris or sclera with or without suture.

Each of these techniques is analyzed and compared below, with the device of the present invention called Arrieta's Holder.

The Arrieta's Holder device offers the possibility of placing an IOL with support in the sclera, which is a better place than in the anterior chamber (AC). The most important difference is that it is behind the iris, where the natural lens of the eye is naturally located. Staying away from the corneal endothelium, which is the structure most frequently affected in cases of IOL implantation in CA.

### Indications for Scleral Fixation of Intraocular Lenses

Scleral fixation of intraocular lenses has its main indication when there is no capsular support or it is defective, making it impossible to place the IOL in the capsular bag, shallow anterior chamber (<3 mm) (contraindication for IOL placement in AC) or corneal conditions such as Fuchs' dystrophy, corneal edema, or already have a corneal transplant.

### Contraindication for IOL Placement in AC

This lack of capsular support can be due to complicated cataract surgery or ocular trauma or a weakness of the capsular support of another origin, in which case they are local or secondary anatomical alterations to systemic diseases, such as Marfan syndrome, Weill-Marchesani, homocystinuria and pseudoexfoliation syndrome (PEX) (by far the most frequent cause of defective capsular support since PEX is characterized by causing a progressive deterioration of the zonule of Zinn (https://es.wikipedia.org/wiki/Ligamento_suspensorio_del_cristalino#:˜:text=The%20ligament%20suspensory20del%20crystalline,di%C3%A1metro% 20de%204%2D50%20%C2%B5m.), being the main alteration in the zonule, which is the one that supports the capsular bag.

Pseudoexfoliation syndrome is the most common risk factor for late intraocular lens dislocation and is estimated to occur in 1.7% of patients undergoing cataract surgery within 25 years after surgery.

On the other hand, an incidence of 1-2% of capsular rupture with vitreous loss is reported in cataract surgery in developed countries, which may contraindicate placement of IOL in the capsular bag. While it is a low incidence, it is by far the most common surgery performed worldwide; therefore, it becomes a significant number.

The common systemic condition associated with weakened zonules and lens subluxation, Marfan syndrome, has an incidence of 1 in 5,000 people.

### Scleral Fixation Techniques, Analysis and Comparison with the Arrieta's Holder of the Present Invention

Several techniques have been described for the scleral fixation of an IOL, among them those that use sutures, those that use glue and finally those that are fixed only within a scleral tunnel.

### Scleral Fixation

To carry out scleral fixation with suture, it is important to take into account external anatomical references such as the limbus, since from this the place where the suture will be placed will be identified. Regarding the techniques: the sutures can be placed from the outside in (external ab) or from the inside out (internal ab).

Regarding suture material, polypropylene (Prolene) is the most widely used, but polyester (Mersilene), polyethylene (Novafil) and polytetrafluoroethylene (Gore-Tex) also provide good results. Different surgical techniques have been described where it is disclosed the creation of flaps, grooves or scleral tunnels to identify and access the ciliary groove and also serve as suture protection. Some PMMA (Polymethylmethacrylate) lens designs have grommets in the haptics, which serve to facilitate suture fixation.

Suture scleral fixation has advantages such as decreased corneal and angular trauma, decreased risk of secondary glaucoma, pupillary blockage, and little or no contact between the IOL and the iris, which reduces the risk of pigment dispersion syndrome and cystic macular edema.

The Arrieta's Holder of the present invention also has all these same advantages of 10Ls fixed to the sclera in its different techniques, because it is also a scleral fixation technique in which the Arrieta's Holder and the IOL are behind the iris and away from the endothelium (where the eye is anatomically designed to have its natural lens in place).

### Technique of Fixation of Lens to Sclera with Suture

Malbran and his colleagues have been credited with the first published description of IOLs attached to the sclera with suture in 1980. A 10-0 polypropylene suture is used to secure the haptics of the IOL to the sclera at the 3-9 o'clock meridians, in a distance of 2 mm posterior to the limbus, concept of external ab suture passes with the use of scleral flaps to cover the suture knots. The concept of introducing the 10-0 polypropylene suture needle into the lumen of a 28 g needle penetrating the eyeball at 180 degrees from the suture needle entrance was introduced. The entry points for both needles are measured 2 mm posterior to the limbus, which gives a more reproducible IOL end position in the ciliary groove compared to internal ab approaches. The suture traverses the eye from groove to groove, and after it was externalized with the use of a second instrument (Sinsky's hook) inserted through a corneal wound, the suture is cut in the middle of this, and fixed to the haptics of the IOL; the lens is then introduced into the eyeball, positioning it behind the iris with the haptics resting in the ciliary groove.

This technique is used today with relatively good success rates. In a study carried out in 2015, with 13 eyes and long-term follow-up that ranged from 5 to 10 years, only two eyes were found with minimal lens decentration that did not affect visual acuity.

The Arrieta's Holder of the present invention has much less possibility of decentration of the IOL and of Tilt (inclination of the IOL with respect to the plane of the iris and the visual axis) of the same, because, unlike the technique of scleral fixation with sutures in which the fixation points to the sclera are only 2 (located at 180 degrees from each other constituting a single fixation axis that is usually on the horizontal axis from 3 to 9 hours), in the case of the Arrieta's Holder of the present invention, there are 3 fixation points to the sclera, located at 120 degrees from each other, constituting three fixation axes, which are usually one horizontal from hour 10 to hour 2 and another two oblique axes, from hour 2 to hour 6, another from hour 6 to hour 10. This allows greater stability of the Arrieta's Holder and the IOL with greater subjection to the sclera and less possibility of decentration or subluxation in the case of any trauma that the patient may have after implantation.

At present there is a tendency to use thicker sutures, such as polypropylene 9-0 and Gore-Tex 7-0 (CV-8) over traditional 10-0 polypropylene, seeking greater durability. Sutures do not usually last more than 10 years, because they are gradually reabsorbed and, in these cases, the IOL loses its fixation to the sclera and can dislocate the vitreous chamber, also causing a decrease in the visual acuity (VA) of the patient because the IOL is no longer in the proper position and probable damage to the retina due to dislocation of the IOL into the vitreous cavity. In these cases of reabsorption of the suture, it is necessary to perform a new surgery on the patient to re-suture the lens and repeat the process as many times as necessary (approximately every 10 years at most and in many cases earlier).

Multiple case series, retrospective analysis, and prospective studies reported late IOL dislocation due to suture rupture secondary to 10-0 polypropylene degradation.

The Arrieta's Holder of the present invention has the advantage that it does not need sutures, since the fixation to the sclera is done through the Arrieta's Supports in both their internal ab and external ab designs.

1. The material of the internal Arrieta's Supports (3) and the external Arrieta's Supports (3bis) is much more noble and biocompatible than any of the existing sutures on the market and lasts much longer than all existing sutures on the market, a proven fact because it is the same material that is used for the manufacture of IOL that has biocompatibility and long-term durability demonstrated by decades of use in millions of patients worldwide.

2. The placement technique is easier than the placement of sutures, with less need for the surgeon's skill in placement.

In 2006, Hoffman (Hoffman, R., Fine, I., & Packer, M. (2006). Scleral fixation without conjunctival dissection. Journal of Cataract & Refractive Surgery, 32 (11), 1907-1912. doi: 10.1016/j. jcrs.2006.05.029) introduced the idea of creating scleral pockets that do not require conjunctival peritomy, but that allow adequate suture coverage. The incisions are started in the cornea 180 degrees apart and using a Crescent knife, dissected in partial thickness in the direction of the sclera and covering a time zone. An anterior paracentesis is done to each of the scleral pockets. Using a 27-gauge transconjunctival needle through the posterior edge of one of the scleral pockets, and at a distance of 1 mm posterior to the surgical limbus, it is inserted into the eyeball. From the opposing paracentesis, one end of the 9-0 double-armed polypropylene on a long straight needle is inserted into the eye and attached to the 27-gauge needle, and the needle along with the suture are externalized through the scleral pocket. The process is repeated with the other end of the polypropylene to capture the haptic. Once the two ends of the suture are externalized through the scleral pocket and conjunctiva, the ends are retrieved from the scleral pocket with a Sinskey hook, and the resulting knot is buried in the scleral pocket as the suture is secured.

This is a variant of the previous Malbran technique in which the suture placement technique changes but all the differences discussed with the placement of the Arrieta's Holder persist.

### Sutureless Lens-to-Sclera Fixation Technique

In sutureless scleral fixation, the IOL haptics are externalized and fixed within the sclera using flaps or scleral tunnels parallel to the limbus. One of the advantages is that three-piece lenses are used without a special design.

While it is true that sutureless techniques have the advantage that they can be done using 3-piece folding 10Ls without a special design in that their cost is much lower than AC IOLs, on the other hand, a disadvantage of sutureless techniques is the fact that Premium single-piece IOLs cannot be used either in their toric, trifocal or extended range versions.

On the other hand, sutureless techniques also have the same disadvantage as suture techniques in the fact that there may be tilt or decentration of the IOLs due to the fact that the fixation points are only two.

The Arrieta's Holder of the present invention also has the advantage that it can be used with a 3-piece IOL as in sutureless techniques, but in addition the Arrieta's Holder can also be used with Premium one-piece IOLs in either its toric and trifocal or extended range versions which are the ones with the best visual result for the patient and also allow the correction of astigmatism and presbyopia of the patient.

The Arrieta's Holder of the present invention has much less possibility of decentration of the IOL and of Tilt (inclination of the IOL with respect to the plane of the iris and the visual axis) of the same, because, unlike the technique of scleral fixation with sutures in which the fixation points to the sclera are only 2 (located at 180 degrees from each other constituting a single fixation axis that is usually on the horizontal axis from 3 to 9 hours), in the case of the Arrieta's Holder of the present invention there are 3 fixation points to the sclera, located at 120 degrees from each other, constituting three fixation axes, which are usually one horizontal from hour 10 to hour 2 and another two oblique axes, from hour 2 to hour 6, another from hour 6 to hour 10. This allows greater stability of the Arrieta's Holder and the IOL with greater subjection to the sclera and less possibility of decentration or subluxation in the case of any trauma that the patient may have after implantation. A sutureless fibrin glue fixation technique has recently been described with short-term visual results comparable to those of sutured scleral fixation. Sutureless scleral fixation seeks to avoid complications associated with suture degradation, scleral erosion, and multiple passes through the sclera and uvea, but long-term studies are still needed.

Scharioth et al. are credited with the first description of the sutureless scleral fixation technique. Sclerotomies diametrically opposite ab external are made at a distance of 1.5-2.0 mm from the limbus. A 24-gauge needle is used to create 50% thick scleral tunnels parallel to the limbus near each of the sclerotomy sites. A standard three-piece IOL is inserted into the eye, the haptics are in turn exteriorized through the sclerotomy incisions and inserted into the scleral tunnels, leaving only a small portion of each haptic exposed between the scleral tunnel and the sclerotomy.

This sutureless scleral placement technique has all the advantages of not requiring sutures that are short-lived and degrade over the years but because the haptics of the 3-piece IOLs were not designed to be placed in the sclera, it has no specific zones for their fixation to the sclera and their long-term stability is under discussion and depends a lot on the technique of creating the scleral tunnel in terms of its design and perfect fit between this scleral tunnel and the haptics of the 3-piece IOLs.

The Arrieta's Holder of the present invention is designed to be fixed to the sclera and has specific areas in the design of the internal Arrieta's Supports (3) and external Arrieta's Supports (3bis) that allow a fixation to the sclera with much greater fixation and does not depend on that the construction technique of the scleral tunnel is of a design that fits perfectly with the haptics of the 3-piece IOL, since it is the internal Arrieta's Supports (3) or external Arrieta's Supports (3bis) that are fixed to the sclera and the IOL is inside the Arrieta's Holder without the need for the IOL to come into contact with the sclera.

### Sutureless 27G Needle Guided Lamellar Scleral Dissection Intrascleral IOL Implantation

Yamane (Karadag, R., & Bayramlar, H. (2014). Re: Yamane et al.: Sutureless 27-gauge needle-guided intrascleral intraocular lens implantation with lamellar scleral dissection (Ophthalmology 2014; 121: 61-6). Ophthalmology, 121 (8), e42. doi: 10.1016/j.ophtha.2014.03.019) describes this technique in 2014. It consists of a 2.8mm sclerocorneal incision and 2 lamellar scleral dissections measuring 1.5 mm in length and approximately 50% scleral thickness at 1.7 mm from the limbus at 180 degrees from each other. A three-piece IOL (Tecnis ZA9003; Abbott Medical Optics, Santa Ana, Calif.) is inserted into the anterior chamber with an injector and the posterior haptic is kept out of the anterior chamber to prevent the lens from sliding into the vitreous cavity. An angled sclerotomy is performed at the end of the lamellar scleral dissection with a 27-gauge needle. The haptic is inserted into the lumen of the needle using forceps. The IOL is rotated and the posterior haptic is inserted into the anterior chamber. A second sclerotomy is then performed with a 27-gauge needle, which is passed through the lamellar scleral dissection 180 degrees from the first sclerotomy. The posterior haptic is introduced into the lumen of the second needle, while the first needle is placed in the eyelid and both haptics are externalized to the sclera with the double-needle technique.

The 2 mm long tunnels parallel to the limbus are made with the 27-gauge needle at the end of the lamellar scleral dissection in front of the sclerotomies. The haptics are inserted into the 1.5 mm tunnels, and the IOL position is centered. A peripheral iridotomy is performed using the vitrectomy cutter to avoid capture by the iris of the IOL. The infusion cannula is removed, and all sclerotomy sites are inspected for wound leaks.

### Intraocular Lens Fixation with Double Needle Technique

In 2017, Yamane (Ganne, P., Baskaran, P., & Krishnappa, N. (2017). Re: Yamane et al.: Flanged intrascleral intraocular lens fixation with double-needle technique (Ophthalmology. 2017; 124: 1136- 1142). Ophthalmology, 124 (12), e90e91. doi: 10.1016/j.ophtha.2017.07.007) modifies his double needle technique to avoid sclerotomy, makes a scleral tunnel using only a 27G needle, with the idea of decrease the risk of postoperative hypotonia. It uses a three-piece lens that inserts into the anterior chamber with an injector and keeps the posterior haptic out of the anterior chamber, to prevent the lens from sliding into the vitreous cavity.

A scleral tunnel is created through the conjunctiva using a 30-gauge needle 2 mm from the limbus.

The main haptic is introduced into the lumen of the needle with microforceps. A second scleral tunnel is made 180 degrees from the first. The second haptic is inserted into the lumen of the second needle holding the first needle fixed. Both haptics are externalized to the conjunctiva at the same time, the ends of the haptics are cauterized to generate a flange with a diameter of 0.3 mm. The ends are cauterized precisely to prevent them from moving out of position because, as mentioned above, the haptics of the 3-piece IOLs do not have any area specifically designed to be fixed to the sclera by placing a cautery very close to the haptic of the 3-piece IOL so that with the heat emanating from the cautery it melts and increases its thickness so that later this thicker part is fixed in the sclera for the fixation of the haptic.

The Arrieta's Holder of the present invention already has an area in the internal Arrieta's Supports (3) and external Arrieta's Supports (3bis) with a specific design for fixation to the sclera with a constant shape and dimensions and not dependent on how much heat is applied to the haptic and how close that heat is and for how long it is applied (all these variables of temperature, distance and time, vary the dimensions of the haptic button that is created in the Yamane technique, in addition to the possibility that this heat source touches the haptic of the 3-piece IOL in which case the cautery remains attached to the haptic and the haptic must be trimmed to continue with the surgery and the consequent change in the size of the haptic that makes the fixation position of the 3-piece IOL is no longer the same as planned from the beginning; the haptic flange is pushed back and fixed in the scleral tunnel.

### Second Haptic—First Modification of Intrascleral Haptic Fixation with Double Needle Technique

The original technique describes introducing the main haptic into the lumen of the first sclerotomy needle. However, securing the main haptic on the needle makes securing the second haptic even more difficult.

Kim made a modification to the technique that involves a rear haptic approach first. 30G gauge needles should be used to improve the ability to fix the haptics. Two needles are prepared, one for the left haptic and one for the right. Each needle is bent with the bevel facing up: the needle on the right side is bent approximately three-quarters in length and the left needle is bent in the center. They are both bent at approximately 80 degrees.

A 1.0 mm inferior limbal incision is made in the VI, III, IX meridian and a 3.0 mm superior one. The IOL is inserted through the upper incision using an injector. The assistant advances the plunger, while the surgeon holds the shaft of the injector. As the haptic emerges from the injector, the surgeon places a 23-gauge microforceps through the lower incision (VI meridian) to grasp the tip of the main haptic.

Subsequently, the right-sided needle is used to pierce the conjunctiva and sclera at mark D, to tunnel through the sclera to mark B, and then pierce the vitreous space.

Through a 1.0 mm limbal incision, located in the III meridian, the secondary haptic is introduced with the microforceps into the lumen of the 30 g needle. The bevel is used as a platform to engage the needle haptic and at least half of the haptic should be inside the needle to prevent it from coming off the needle. With the left needle, the scleral tunnel is made in the same way as on the right and continuing from point C to A where the needle is rectified to enter the vitreous cavity. From the IX paracentesis with the forceps, the haptic is taken and inserted into the lumen of the needle. Now both 30 g needles are externalized at the same time, externalizing the haptics to the subconjunctival space. The hand cautery is used to create a terminal bulb to prevent them from sliding into the vitreous space, leaving them at the entrance to the scleral tunnel.

Instead of sutures, fibrin glue has also been used to attach the haptics of an IOL to the sclera. This technique was first described in 2008 by Agarwal et al. and involves the creation of two scleral flaps 180 degrees apart. Sclerotomies are performed within the flaps and, after the introduction of the IOL into the eye, the haptics are grasped through the sclerotomies with forceps and externalized. Fibrin glue is then applied to the flap bed, and the outer portion of the scleral flap is folded over the haptic, sealing the scleral flap.

This technique also has all the advantages that we describe of not needing sutures that have a short duration and degrade over the years, but since the haptics of the 3-piece IOLs were not designed to be placed in the sclera, it does not have specific areas to their fixation to the sclera and their long-term stability is under discussion and depends a lot on the technique of creating the scleral tunnel in terms of its design and perfect fit between this scleral tunnel and the haptics of the 3-piece IOLs and how long the glue used lasts and the adhesion and resistance it has to be able to fix the haptic of the 3-piece IOL.

The Arrieta's Holder of the present invention is designed to be fixed to the sclera and has specific areas in the design of the internal Arrieta's Supports (3) and external Arrieta's Supports (3 bis) that allow fixed to the sclera with much greater fixation and does not depend on the scleral tunnel construction technique being of a design that fits perfectly with the haptics of the 3-piece IOL because it is the internal Arrieta's Supports (3) or external Arrieta's Supports (3 bis) that are fixed to the sclera and the IOL goes inside the Arrieta's Holder without the need for the IOL to come into contact with the sclera, and without the need to use adhesives to fix it.

The Arrieta's Holder of the present invention has much less possibility of decentration of the IOL and of Tilt (inclination of the IOL with respect to the plane of the iris and the visual axis) of the same, because, unlike the technique of scleral fixation with sutures in which the fixation points to the sclera are only 2 (located at 180 degrees from each other constituting a single fixation axis that is usually on the horizontal axis from 3 to 9 hours), in the case of the Arrieta's Holder of the present invention, there are 3 fixation points to the sclera, located at 120 degrees from each other, constituting three fixation axes, which are usually one horizontal from hour 10 to hour 2 and another two oblique axes, from hour 2 to hour 6, another from hour 6 to hour 10. This allows greater stability of the Arrieta's Holder and the IOL with greater subjection to the sclera and less possibility of decentration or subluxation in the case of any trauma that the patient may have after implantation.

In a recent study of 25 eyes that underwent fibrin glue-assisted fixation, there was IOL decentration in only one eye (4%) after one year of follow-up.

In 2013, Agarwal (Agarwal, A., Jacob, S., Kumar, D., Agarwal, A., Narasimhan, S., & Agarwal, A. (2013). Handshake technique for glued intrascleral haptic fixation of a posterior chamber intraocular lens. Journal Of Cataract & Refractive Surgery, 39 (3), 317-322. doi: 10.1016/j.jcrs.2013.01.019) describes a variation of his technique defined as “handshake”, which stands out for be a technique that facilitates the externalization of the haptics for intrascleral fixation.

The “handshake” technique is a modification that provides better intraocular maneuverability throughout surgery and extends the applicability of the technique for challenging cases requiring haptic manipulation, and the intraoperative advantage of a well-formed balloon throughout surgery.

### Complications

Sutureless techniques attempt to avoid suture-related complications by burying the loops in tunnels or scleral flaps. However, these lenses can still dislocate, and although good short-term results are described, long-term data on lens stability and the rate of serious complications are lacking.

The Arrieta's Holder of the present invention has a specific area designed for scleral fixation, with which the long-term stability of the IOL is much greater than with existing techniques up to now.

### Conclusion

Surgical techniques for lenses fixed to the sclera are improving as surgeons describe modifications to existing techniques, with the aim of reducing perioperative complications associated with suture placement, reducing the risk of lens decentration and subluxation.

Also, as the industry improves suture options and lens designs improve, surgeons will have more options to improve outcomes and reduce postoperative complications. At the moment, it is an excellent option for those cases in which these techniques are required.

This is precisely the case of the Arrieta's Holder of the present invention, a new design of a support for the placement of IOLs in cases where the capsular support is not good enough to place an IOL in the capsular bag.

It has the advantage of being a scleral support design that is more modern in IOL support techniques and with better visual results, long-term stability and reduced risks and complications compared to the other techniques described above.

In addition to this, it does not need sutures or glues that have a short duration in time and less fixation capacity and stability in the fixation to the sclera.

In summary, the scleral fixation device for intraocular lens support (Arrieta's Holder) of the present invention, unlike the other scleral support techniques:

1. allows the placement of both 3-piece IOLs (the only type of IOL that can be used in scleral support techniques known to date) and single-piece IOLs.

2. allows the placement of Premium toric IOLs (toric IOLs cannot be placed with the other techniques since 3-piece IOLs do not come in toric versions).

3. allows the placement of trifocal or extended range IOLs (trifocal or extended range IOLs cannot be placed as 3-piece folding 10Ls do not come in trifocal or extended range versions).

4. in the case of zonular weakness that occurs during cataract surgery or in cases that already existed previously, but the surgeon notices it during cataract surgery, the IOL chosen for that patient should usually be changed for another folding IOL of 3-piece (if the surgeon does not have that other 3-piece folding IOL with the exact measurement for that patient at the time of surgery, a second surgical act should be planned at another date when the appropriate 3-piece folding IOL is obtained for the patient). In the case of having the Arrieta's Holder of the present invention, the surgeon can use the same IOL that he had planned to use with that patient and resolve the situation in the same surgical act and not have to wait for another date to perform a second surgery to place the 3-piece folding IOL fixed to the sclera.

That the surgeon can count on a second folding IOL with 3 spare parts at the time of surgery with the same diopter that the patient needs, implies having a stock of IOLs of many different sizes on hand in the operating room, which in turn implies a very high cost in supplies intended only for those occasional cases in which it is necessary to use these 3-piece folding IOLs and it also continues to be an alternative IOL, which is not the one that the surgeon chose for that patient, due to various particular characteristics of that case.

### BIBLIOGRAPHY

1. Kim, E., Brunin, G., & Al-Mohtaseb, Z (2016). Lens Placement in the Absence of Capsular Support. International Ophthalmology Clinics, 56 (3), 93-106. doi: 10.1097/iio.0000000000000116
2. Stem, M., Todorich, B., Woodward, M., Hsu, J., & Wolfe, J. (2017). Scleral-Fixated Intraocular Lenses. Journal Of Vitreoretinal Diseases, 1 (2), 144-152. Doi: 10.1177/2474126417690650
3. Kim, D. (2018). Trailing-haptic-first modification of double-needle intrascleral haptic fixation technique. Journal Of Cataract & Refractive Surgery, 44 (4), 424-428. Doi: 10.1016/j.jcrs.2018.01.027

### BRIEF DESCRIPTION OF THE DRAWINGS

These Figures are included by way of illustration and do not limit the present invention in any way.

FIG. 1: Perspective view of the human eye with the Arrieta's Holder in place.

FIG. 2: Right side view of the human eye with the Arrieta's Holder in place.

FIG. 3: Perspective view of the Arrieta's Holder with its Arrieta's Support deployed.

FIG. 4: Front, side T-T view of the Arrieta's Holder, Section T-T of the Arrieta's Holder with its Arrieta's Internal Supports deployed and Detail U scale 10:1.

FIG. 5: Front, side and bottom view of the Arrieta's Support of the Arrieta's Holder with sectional view L-L.

FIG. 6: Perspective view of the Arrieta's Support of the Arrieta's Holder.

FIG. 7: Perspective view of the Arrieta's Support of the Arrieta's Holder.

FIG. 8: Front, side and bottom view of the Arrieta's Support positioning device of the Arrieta's Holder.

FIG. 9: Perspective view of the positioning device of the Arrieta's Support of the Arrieta's Holder.

FIG. 10: Additional perspective view of the positioning device of the Arrieta's Support of the Arrieta's Holder.

FIG. 11: Front view and I-I section and bottom view with J-J section of the Arrieta's.

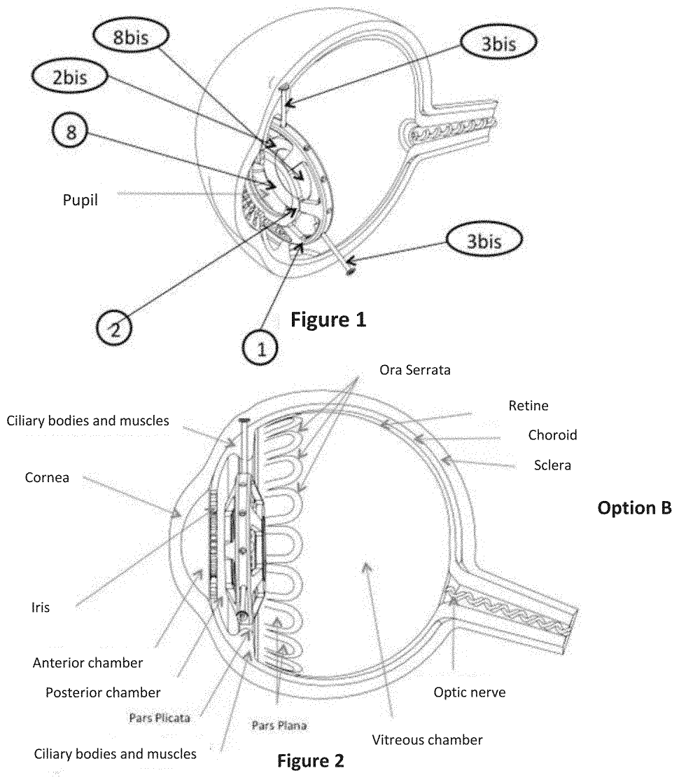

### DETAILED DESCRIPTION OF THE INVENTION

The scleral fixation device for intraocular lens support (Arrieta's Holder) of the present invention comprises:

an Arrieta's Holder receptacle itself and

a plurality of Arrieta's Supports from receptacle to sclera.

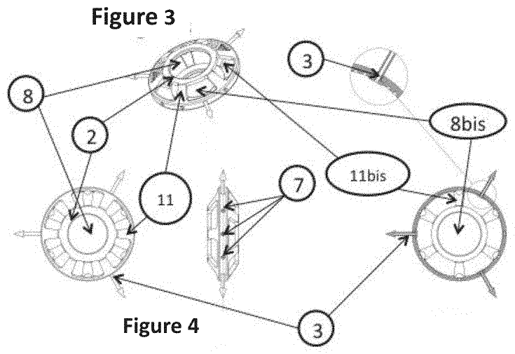

Said Arrieta's Holder is characterized by being a receptacle where the IOL (intraocular lens) is contained, where said receptacle (1) consists of two faces facing each other, the anterior face (2) and posterior face (2bis) which are joined by an equatorial ring (10). See FIGS. 3 and 4.

The faces (2) and (2bis) are joined in the ring (10) in such a way as to define a cavity (4), which will contain the IOL. See FIGS. 3 and 4.

The faces (2) and (2bis) will have an anterior central hole (8) and a posterior central hole (8bis) in such a way as to allow the passage of light so that the IOL can fulfill its function.

For construction purposes, faces (2) and (2bis) in the space occupied between their outer perimeter and the central hole, have anterior connecting bridges (11) and posterior connecting bridges (11bis), such as those seen in FIGS. 3, 4.

In the equatorial ring (10) there are at least twelve perforations (7), which allow the fixation of the Internal Arrieta's Supports (3) and External Arrieta's Support (3bis). See FIGS. 3 and 4.

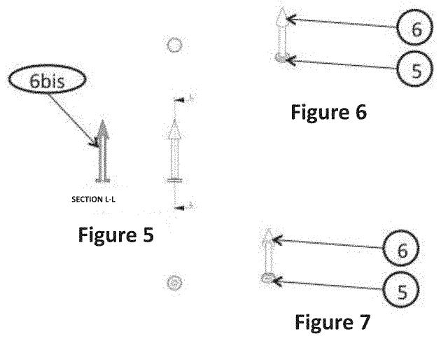

The Internal Arrieta's Supports (3) and External Arrieta's Support (3bis) have a widened disk-shaped shape at one end (5) and an arrow-shaped shape at the other end (6). See FIGS. 5 and 6.

The fixation of the Internal Arrieta's Supports (3) and External Arrieta's Support (3bis) (see FIGS. 5 and 6) in the Arrieta's Holder device (1) can be done at either of its two ends.

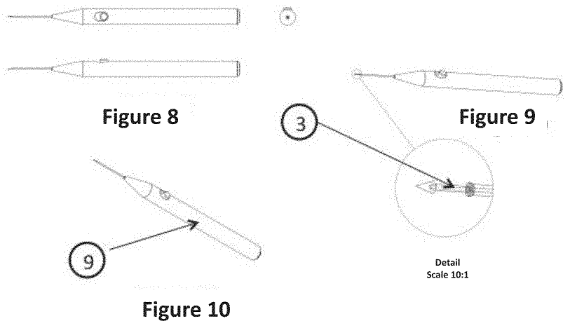

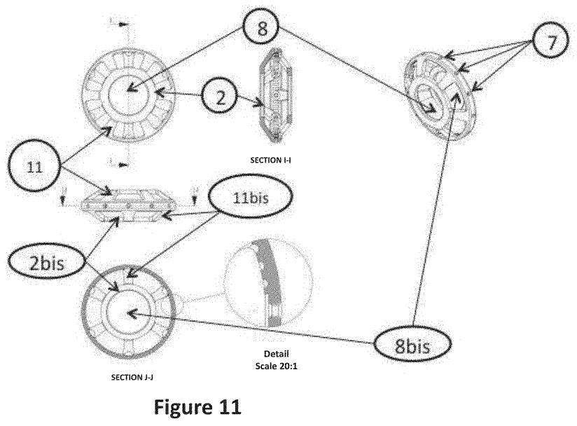

FIG. 8 refers to the placement device (9) of the Internal Arrieta's Support (3) and External Arrieta's Support (3bis). It is a device that has an internal metal stem (6bis) (see FIGS. 5 and 6), which guides the Arrieta's Support; when the Arrieta's Support is loaded in the injector, the stem is inside the internal conduit of the Arrieta's Support and serves as guide for the injection of the same. Once the Arrieta's Support is placed in the desired position, the stem is withdrawn from the Arrieta's Support through a recoil of the same that occurs when we press on the supports, which are placed on the tips of the index and thumb fingers in the time of injection. Said placement device (9) is outside the scope of the present invention and is mentioned for the purpose of fully understanding the placement procedure.

The Arrieta's Holder of the present invention is made of polymethacrylate, silicone, acrylic or other material suitable for insertion into the human body and has dimensions that range from 10 to 14 mm in length and 3 to 6 mm in thickness.

The method for its placement in the patient's eye is as follows:

### Placement of the Arrieta's Holder Ab Internal (from Inside to Outside)

30 G valved trocars are placed to perform a vitrectomy of the entire vitreous base and then the irrigation is left on to maintain the 10P at an optimal value.

3 paracentesis of 1mm are performed at hours 1, 4 and 8.

A 2 mm corneal incision is made between 10 and 11 hours, through which the Arrieta's Holder of the present invention will be injected.

Viscoelastic is placed in the Anterior Chamber to protect the endothelium.

The Arrieta's Holder is injected through the 2.2 mm corneal incision, through which the Arrieta's Holder injector is introduced to be positioned at the level of the plane of the iris and is then temporarily held from its distal end with forceps through of the 1 mm paracentesis at hour 1. The injector of the internal Arrieta's Supports (3) is inserted with the same placed at its distal end, through the 2.2 mm corneal incision and the most distal hole of the Arrieta's Holder is threaded with the internal Arrieta's Support (3) to then enter the scleral thickness, crossing the ciliary body in the valleys of its folds. Once the internal Arrieta's Supports (3) have reached a depth of 90% of the scleral thickness, the internal Arrieta's Support (3) is released so that it is fixed to the sclera by its umbrella shape that prevents recoil and its basal end remains supporting the Arrieta's Holder at the level of the injection hole because its base is wider than its body and wider than the holes of the Arrieta's Holder.

Then the procedure is repeated to place two more supports, all at the same depth and each preferably at 120 degrees from the previous one with the same maneuver. The quantity and angular arrangement of the Arrieta's Supports is at the discretion of the surgeon.

To place the internal Arrieta's Support (3) of hour 2, the forceps are introduced through the paracentesis at hour 8 and to place the internal Arrieta's Support (3) of hour 10 the paracentesis of hour 4 is extended to 2.2 mm to insert the internal Arrieta's Support (3) and the forceps is held through the 1-hour paracentesis. With this, the Arrieta's Supports are fixed to the sclera in three equidistant areas and fixed to the sclera by internal ab route, without reaching to have communication with the outside, to avoid being a gateway to germs and not causing erosion of the conjunctiva by decubitus.

It is sufficient to place only three internal Arrieta's Supports (3) but six or more can be placed as desired by the surgeon. The idea is that these internal Arrieta's Supports (3) are not removed but if it is necessary to remove the Arrieta's Holder, the base of the Arrieta's Supports can be cut off, thereby freeing it to extract the Arrieta's Holder or to place it in another position, if necessary.

### Placement of the Arrieta's Holder ab External (from Outside to Inside)

A 3 mm wide opening of the conjunctiva is made 2 mm from the limbus at 10, 2, and 6 hours. 3 scleral pockets are created 2 mm from the limbus, 2 mm wide by 2 mm long and at a depth of 400 um at hours 10, 2, and 6.

30 G valved trocars are placed to perform a vitrectomy of the entire vitreous base and then the irrigation is left on to maintain the IOP at an optimal value.

3 paracentesis of 1mm are performed at hours 1, 4, and 8.

A 2.2 mm corneal incision is made between 10 and 11 hours, through which the Arrieta's Holder will be injected.

Viscoelastic is placed in the anterior chamber to protect the endothelium.

The Arrieta's Holder is injected through the 2.2 mm corneal incision, with the Arrieta's Support injector to be positioned at the level of the iris plane and then temporarily held from its distal end with a forceps through the paracentesis of 1 mm of hour 1. The injector of the external Arrieta's Supports (3bis) is introduced with the same placed in its distal end, through the scleral pocket of hour 6, the sclera is perforated towards the vitreous cavity with the injector that has a 30 G metal shaft with a sharp tip to pierce the sclera and then the external Arrieta's Support (3bis) is inserted into the 6 hour hole of the Arrieta's Holder, which is held by the 1 hour forceps, with this the external Arrieta's Support (3bis) remains fixed to the Arrieta's Holder by the umbrella shape that prevents its backward movement.

The external Arrieta's Support (3bis) is released, which remains fixed to the sclera by its wider base that prevents it from deepening, leaving this base in the scleral pocket that was previously constructed.

Then the procedure is repeated to place two more supports, all at the same depth and each one, preferably at 120 degrees from the previous one with the same maneuver. The quantity and angular arrangement of the Arrieta's Supports are at the discretion of the surgeon.

To place the external Arrieta's Supports (3bis) at hour 2, the forceps are introduced through the paracentesis at hour 8 and to place the external Arrieta's Supports (3bis) at hour 10, the paracentesis at hour 4 is used. Thus, the Arrieta's Holder is fixed to the sclera in three equidistant areas and fixed to the sclera by external ab via and then the conjunctiva is closed with 8.0 silk.

It is enough to place only three external Arrieta's Supports (3bis) but six or more can be placed as desired by the surgeon. The idea is that these external Arrieta's Supports (3bis) are not removed but if it is necessary to remove the Arrieta's Holder, the base of the external Arrieta's Supports (3bis) can be cut, which would be released to extract the Arrieta's Holder or to place it in another position, if necessary.

Among the advantages and solutions provided by the device of the present invention, the following can be mentioned:

1. It does not cause a decrease in corneal endothelial cells due to the proximity or contact of the IOL with the endothelium as occurs with the Anterior Chamber IOLs.

2. It does not cause iris atrophy in the IOL fixation area, since it is not fixed to iris but to sclera, as occurs with AC IOLs in its Iris-Claw models, either in its versions fixed in the anterior iris face or its version fixed in the posterior iris face.

3. It does not cause ovalizations of the pupil (cat's eye), since it is not fixed to iris but to sclera, as occurs in the case of AC IOLs in its Iris-Claw models, either in its versions fixed in the anterior iris face or its version fixed in the posterior iris face.

4. In the case of zonular weakness that occurs during cataract surgery or in cases that already existed previously, but the surgeon notices it during cataract surgery, the IOL chosen for that patient should usually be changed for another Anterior Chamber AC IOL. If the surgeon does not have that other AC IOL with the exact measure for that patient at the time of surgery, a second surgical act should be planned at another date when the appropriate 3 AC IOL is obtained for the patient. In the case of having a device of the present invention (Arrieta's Holder), the surgeon can use the same IOL that he had planned to use with that patient and resolve the situation in the same surgical act, and not have to wait for another date to do a second surgery to place the iris fixed AC IOL. In addition, for the surgeon to have a second replacement AC IOL at the time of surgery with the same diopter required by the patient, it implies having a stock of IOLs of many different sizes on hand in the operating room, which in turn implies a very high cost in supplies destined only for those occasional cases in which it is necessary to use these AC IOLs, and it also continues being an alternative IOL, which is not the one that the surgeon chose to use for that patient, due to various particular characteristics of that case.

5. IOLs fixed to the sclera have a theoretical advantage over other techniques with respect to complications, especially in eyes with a history of trauma and in young patients.

1. A scleral fixation device for an intraocular lens support comprising:
a receptacle, and
a plurality of clamping arms the receptacle to the sclera.

2. The device of claim 1, wherein the receptacle includes:
an anterior part shaped as a circumference,
a posterior part shaped as a circumference, and
an equatorial ring located between the anterior part and the posterior part, the anterior part is placed on the front side of the device and posterior part is placed on the back side of the device, the anterior and the posterior parts act as covers to the equatorial ring: wherein the anterior part and the posterior part include a perforation on a center point; an intraocular lens housed in adefined space between the anterior and posterior parts.

3. The device of claim 2, wherein the equatorial ring has, radially on its edges, a plurality of holes in which the damping arms are inserted.

4. The device of claim 3, wherein the clamping arms have an elongated shape with a widening of its section at the proximal end for the purposes of insertion and retention in the equatorial ring, and another widening in its section at the distal end, to be fixed in the sclera preventing radial or axial displacement of the intraocular device.

5. The device of claim 4, characterized in that the Arrieta's Supports have their ends with a flattened disk shape, conical shape or other shape that allows their fixation to the eye tissue.

6. (canceled)

7. The device of claim 16, wherein the intraocular lens (IOL) is a monofocal, a multifocal, or a toric multifocal lens.

8. A surgical method of inserting the device of claim 1, the method is performed by an internal ab route according tothe following steps:
a placeinge 30 G valved trocars to perform a vitrectomy of the entire vitreous base and then leave the irrigation on so that the intraocular pressure is maintained at an optimal value;
b. performing 3 paracentesis of 1 mm at hours 1, 4, and 8;
c. makinge a 2 mm corneal incision between 10 and 11 hours, through which the intraocular device will be injected;
d. placnning viscoelastic in the anterior chamber to protect the endothelium;
e. injecting the intraocular device through the 2.2 mm corneal incision, through which the injector of the intraocular device is introduced to be positioned at the level of the iris plane and then temporarily hold it at its distal end with forceps through the 1-hour 1-mm paracentesis;
f. inserting the injector of the internal Arrieta's Supports with them placed at its distal end, through the 2.2 mm corneal incision and thread the most distal hole of the intraocular support with the internal Arrieta's Support made of PMMA (polymethacrylate) and then insert it in the scleral thickness, crossing the ciliary body in the valleys of its folds,
g. once the internal Arrieta's Supports have reached a depth of 90% of the scleral thickness, release the internal Arrieta's Support so that it is fixed to the sclera at its widened or umbrella-shaped distal end that prevents recoil and where its proximal end is holding the intraocular device at the level of the injection hole because its base is wider than its body and wider than the holes of the intraocular device,
h. repeating the procedure to place at least two more Arrieta's Supports, all at the same depth and each one, preferably 120 degrees from the previous one with the same maneuver; to place the internal Arrieta's Support at hour 2, the forceps are inserted through the paracentesis at hour 8, and to place the internal Arrieta's Support for hour 10, the paracentesis at hour 4 is extended to 2.2 mm to introduce the injector of the internal Arrieta's Support and the forceps is held through the 1 hour paracentesis, with which the intraocular device is fixed to the sclera in three equidistant areas and fixed to the sclera by internal ab route, without communicating with the outside, to avoid being a gateway to germs and not cause erosion of the conjunctiva by decubitus.

9. A surgical method of inserting the device of claim 1, the method is performed by an external ab route according to the following steps:
a. making opening of the conjunctiva 3 mm wide at 2 mm from the limbus at 10, 2, and 6 hours;
b. creating 3 scleral pockets at 2 mm from the limbus, 2 mm wide by 2 mm long and at a depth of 400 um at hours 10, 2, and 6;
c. placing 30 G valved trocars to perform a vitrectomy of the entire vitreous base and then leave the irrigation on so that the intraocular pressure is maintained at an optimal value;
d. performing 3 paracentesis of 1 mm at hours 1, 4, and 8;
e. making a a 2.2 mm corneal incision between 10 and 11 hours, through which the intraocular device will be injected;
f. place viscoelastic in the anterior chamber to protect the endothelium;
g. injecting the intraocular device through the 2.2 mm corneal incision, with the intraocular device injector to be positioned at the level of the iris plane and then transiently hold its distal end with a forceps through the 1 mm paracentesis at hour 1;
h. inserting the injector of the external Arrieta's Supports with it placed at its distal end, through the 6 hour scleral pocket, drilling the sclera into the vitreous cavity with the injector that has a 30 G metal shaft with a sharp point for drilling the sclera and then introduce the external Arrieta's Support into the 6-hour hole of the intraocular device, which is held by the 1-hour forceps, with which the external Arrieta's Support is fixed to the intraocular device by the umbrella shape that prevents it from recoiling;
i. releasnge the external Arrieta's Support, which remains fixed to the sclera by its wider base that prevents it from deepening, leaving this base in the scleral pocket that was previously constructed;
j. repeating the procedure to place at least two more arms, all at the same depth and each one, preferably 120 degrees from the previous one with the same maneuver,
k. placing the external Arrieta's Support at hour 2, the forceps are inserted through the paracentesis at hour 8 and to place the external Arrieta's Support at hour 10, the paracentesis of hour 4 is used, with which the intraocular device is fixed to the sclera in three areas and equidistantly fixed to the sclera via the external ab route and then the conjunctiva is closed with 8.0 silk.
